(19) United States
(12) Patent Application Publication
(10) Pub. No.: US 2012/0296294 A1
(43) Pub. Date: Nov. 22, 2012

(54) ABSORBENT STRUCTURE FOR BODILY FLUID-ABSORBENT WEARING ARTICLE

(75) Inventors: Tatsuya Hashimoto, Kanonji-shi (JP); Toshifumi Otsubo, Kanonji-shi (JP); Mariko Yamashita, Kanonji-shi (JP)

(73) Assignee: UNICHARM CORPORATION, Ehime (JP)

(21) Appl. No.: 13/577,037

(22) PCT Filed: Feb. 16, 2011
(86) PCT No.: PCT/JP2011/000859

§ 371 Date: Aug. 3, 2012

(30) Foreign Application Priority Data
Feb. 16, 2010 (JP) 2010-031836

(51) Int. Cl. A61F 13/53; A61F 13/51

(52) U.S. Cl. 604/367; 604/385.01

(57) ABSTRACT

The present invention to provide an absorbent structure adapted to prevent the liquid-absorbent materials from falling off the wrapping sheets. An aggregate of liquid-absorbent materials in an absorbent structure has an inner surface covered with a first wrapping sheet and an outer surface and lateral surfaces covered with a second wrapping sheet. The first wrapping sheet and the second wrapping sheet respectively have first extended portions lying outboard of the lateral surfaces and second extended portions wherein the first extended portions and the second extended portions are put flat and bonded together to form laminated regions. The laminated regions cover the lateral surfaces of the aggregate and part of the outer surface which, in turn, covers these lateral surfaces and the outer surface.

### TECHNICAL FIELD

The present disclosure relates to absorbent structures for bodily fluid-absorbent wearing articles.

### RELATED ART

Conventionally, bodily fluid-absorbent wearing articles such as disposable diapers are each provided with an absorbent structure, which comprises a liquid-pervious inner sheet lying on a skin-facing side, an outer sheet lying on a garment-facing side and an aggregate of liquid-absorbent materials sandwiched between such inner sheet and outer sheet.

For example, in the absorbent structure disclosed in JP 2008-154775 A (PATENT DOCUMENT 1), an absorbent core is formed of an aggregate of hydrophilic fibers and absorbent polymer particles and upper and lower wrapping sheets are respectively bonded to upper and lower surfaces of the absorbent core with adhesives to cover the absorbent core. The lower wrapping sheet extends outward from side edges of the absorbent core and is folded to cover the side edges of the core also. The portions of the lower wrapping sheet extending outward and folded are bonded to the upper wrapping sheet along regions in which the lower wrapping sheet overlaps the upper wrapping sheet. As the upper and lower wrapping sheets, tissue papers or liquid-pervious nonwoven fabrics are used.

### CITATION LIST

### Patent Literature

[PATENT DOCUMENT 1] JP 2008-154775 A

### SUMMARY OF INVENTION

### Problem to be Solved by the Invention

When fibers each having a relatively short fiber length and polymer particles each having a relatively small particle diameter are used as liquid-absorbent materials for an absorbent structure, as an important component in a bodily fluid-absorbent wearing article, and such liquid-absorbent materials are covered with porous wrapping sheets, the liquid-absorbent materials might fall through fine pores of the wrapping sheets and/or the wrapping sheets might partially burst, causing the liquid-absorbent materials to fall out. The liquid-absorbent materials, having fallen out, may cling to the wearer's skin and/or disfigure the wearing article. Such situation is undesirable not only for the wearer but also for the manufacturers of the wearing articles. Therefore, it is desirable to provide an absorbent structure adapted to prevent the liquid-absorbent materials from falling out from the wrapping sheets.

### Measure to Solve the Problem

An absorbent structure is provided comprising an aggregate of liquid-absorbent materials covered with a liquid-pervious first wrapping sheet lying on a skin-facing side and a liquid-pervious or liquid-impervious second wrapping sheet lying on a garment-facing side.

One or more aspects of the present invention are characterized in features as will be described below: the absorbent structure has a longitudinal direction, a transverse direction and a thickness direction being orthogonal one to another and includes at least hydrophilic fibers as a liquid-absorbent material. The aggregate has an inner surface lying on the side of the first wrapping sheet as viewed in the thickness direction, an outer surface lying on the side of the second wrapping sheet and lateral surfaces extending in the longitudinal direction on both sides as viewed in the transverse direction to connect the inner surface with the outer surface. The first wrapping sheet covers the inner surface and includes first extended portions extending in the transverse direction beyond the lateral surfaces so as to lie outboard of the aggregate and the second wrapping sheet covers the outer surface and the lateral surfaces and includes second extended portions extending in the transverse direction beyond the lateral surfaces so as to lie outboard of the aggregate. The first and second wrapping sheets are put flat and bonded together in the first and second extended portions to form laminated regions. The laminated regions are curved downward as viewed in the thickness direction to cover the lateral surfaces and part of the outer surface of the aggregate, from the outside of the second wrapping sheet.

### BRIEF DESCRIPTION OF DRAWINGS

FIG. 1 is a perspective view of a disposable pant-type diaper as one example of bodily fluid-absorbent wearing articles.

FIG. 2 is a partially cutaway plan view showing the diaper of FIG. 1 as has been flatly developed.

FIG. 3 is an exploded perspective view of the flatly developed diaper of FIG. 2.

FIG. 4 is a sectional view of the diaper taken along the line IV-IV in FIG. 3.

FIG. 5 is a sectional view of the diaper taken along the line V-V in FIG. 2.

FIG. 6 is a diagram illustrating several steps for production of the absorbent structure.

FIG. 7 is a diagram illustrating the steps as illustrated by FIG. 6 and subsequent steps.

### DETAILED DESCRIPTION OF THE PREFERRED EMBODIMENTS

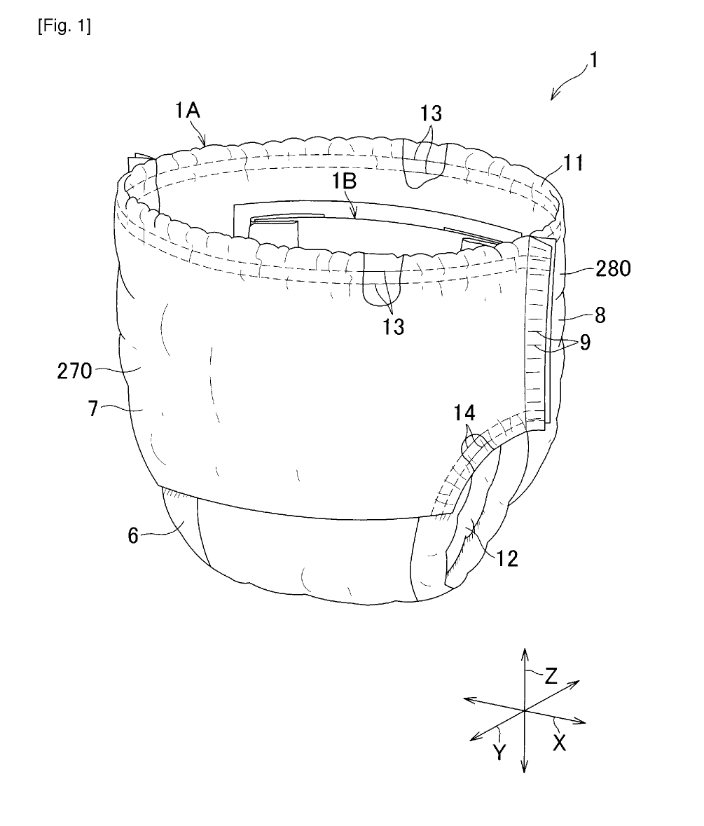

The diaper 1 comprises a chassis 1A of pant-shape and a inner member 1B contractively attached to the inner surface of the chassis 1A wherein a plurality of waist elastic members 13 are attached under tension to the chassis 1A along a periphery of a waist-opening 11. In the vicinity of leg-openings 12, a front panel 270 and a rear panel 280 respectively defining front and rear waist regions 7, 8 and peripheral edges of upper halves of the respective leg-openings 12 are provided with a plurality of leg elastic members 14 contractively attached thereto under tension. In FIG. 1, a transverse direction, a front-back direction and a vertical direction of the diaper 1 are denoted by double-headed arrows X, Y and Z.

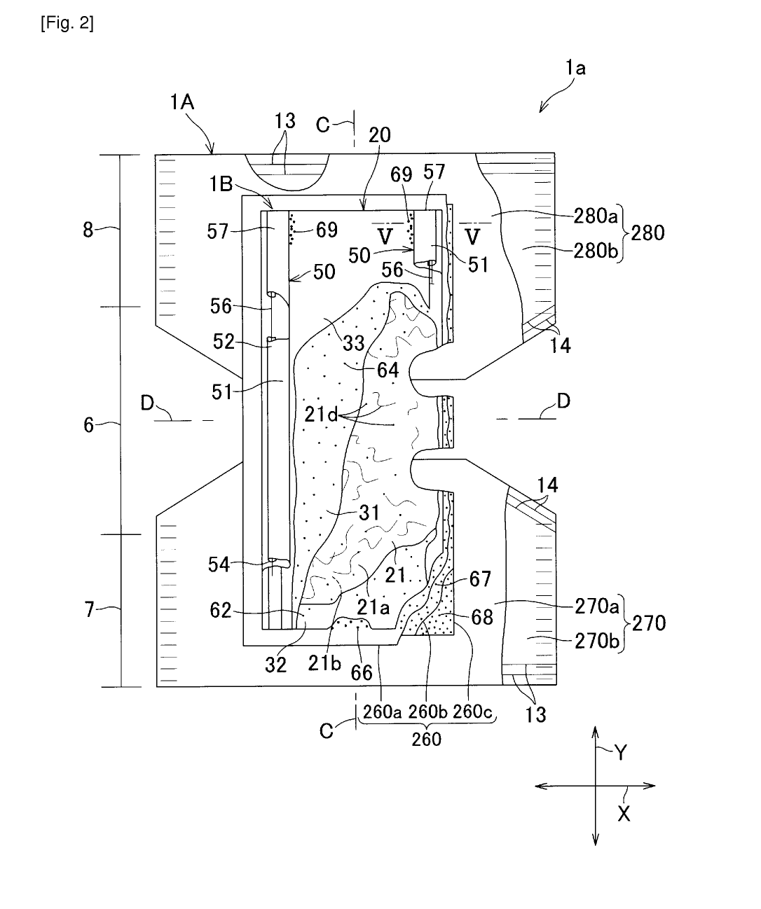

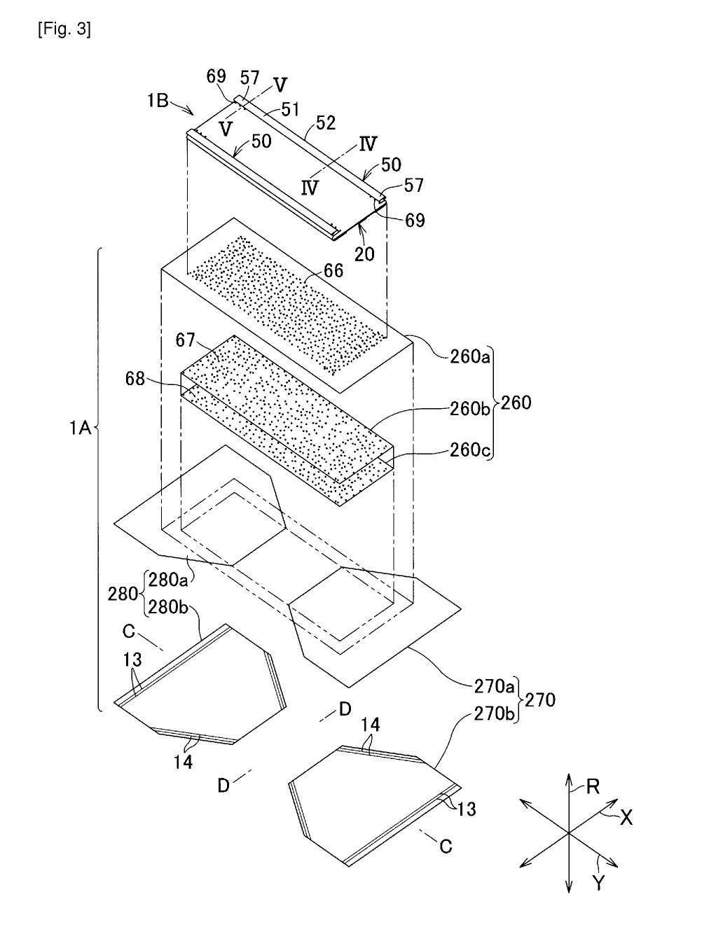

FIGS. 2 and 3 indicate the longitudinal center line C-C in the front-back direction Y bisecting a transverse dimension of the developed diaper 1a in the transverse direction X and the transverse center line D-D in the transverse direction X extending orthogonally to the longitudinal center line C-C so as to bisect a dimension of the developed diaper 1a in the front-back direction Y. The double-headed arrows X, Y denote the transverse direction and the front-back direction of the developed diaper 1a just like these double-headed arrows X, Y denote in FIG. 1. It should be appreciated that, in the description with reference to FIGS. 2 and 3, the direction denoted by the double-headed arrow Y will be sometimes reworded by a longitudinal direction of the chassis 1A and the inner member 1B. A double-headed arrow R in FIG. 3 denotes a thickness direction of the developed diaper 1a.

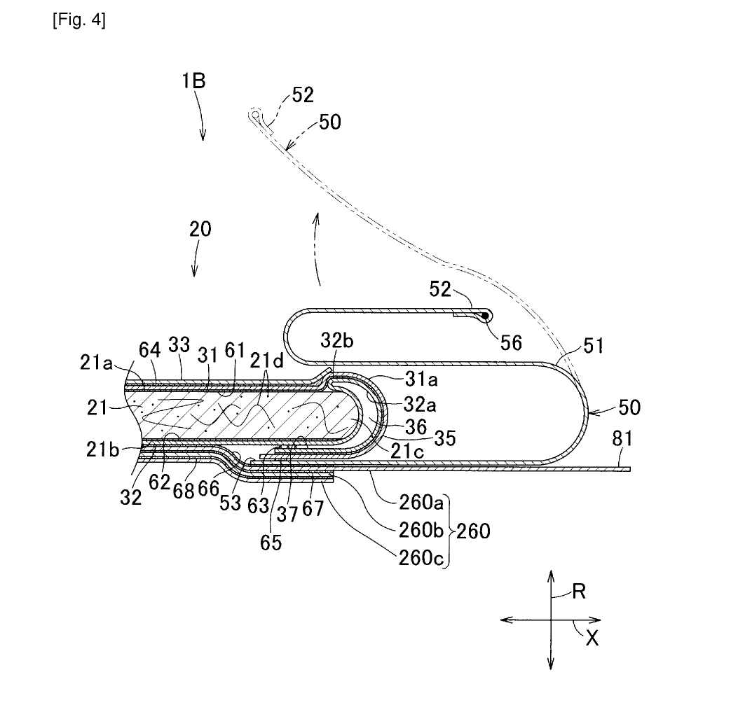

Referring to FIGS. 2 and 3, the chassis 1A comprises a generally hexagonal front panel 270 defining the front waist region 7 and part of the crotch region 6, a generally hexagonal rear panel 280 defining the rear waist region 8 and part of the crotch region 6 and a rectangular center panel 260 defining part of the crotch region 6. The front panel 270 comprises, in addition to an inner sheet 270a and an outer sheet 270b, the waist elastic members 13 and the leg elastic members 14 both sandwiched between these sheets 270a, 270b. While the inner sheet 270a and the outer sheet 270b are the same in shape as well as in size in the illustrated embodiment, it is possible to use the inner sheet 270a and the outer sheet 270b differing from each other in shape and/or in size to form the front panel 270. The inner sheet 270a and the outer sheet 270b are respectively formed of liquid-pervious nonwoven fabrics and moisture-pervious but liquid-impervious plastic films bonded to each other with hot melt adhesives (not shown). The rear panel 280 comprises, in addition to an inner sheet 280a and an outer sheet 280b, the waist elastic members 13 and the leg elastic members 14 sandwiched between these sheets 280a, 280b. While the inner sheet 280a and the outer sheet 280b are the same in shape as well as in size in the illustrated embodiment, it is possible to use the inner sheet 280a and the outer sheet 280b differing from each other in shape and/or in size to form the rear panel 280. The inner sheet 280a and the outer sheet 280b are formed of liquid-pervious nonwoven fabrics and moisture-pervious but liquid-impervious plastic films bonded to each other with hot melt adhesives (not shown). The center panel 260 comprises an inner sheet 260a, an intermediate sheet 260b and an outer sheet 260c. In a preferred center panel 260, dimensions of the inner sheet 260a in the transverse direction X as well as in the longitudinal direction Y are larger than those of the intermediate sheet 260b and the outer sheet 260c. While the intermediate sheet 260b and the outer sheet 260c are substantially the same in shape, the outer sheet 260c is slightly larger than the intermediate sheet 260b. In the developed diaper 1a of FIG. 2, such inner sheet 260a defines both lateral edges of the crotch region 6. In the preferred center panel 260, the inner sheet 260a and the outer sheet 260c are formed of liquid-pervious nonwoven fabrics having a comfortable texture and the intermediate sheet 260b is formed of a liquid-impervious plastic film. The inner sheet 260a, the intermediate sheet 260b and the outer sheet 260c are bonded one to another along regions in which these sheets overlap one another with seventh and eighth hot melt adhesives 67, 68 (See FIG. 4). The inner member 1B is bonded to the inner surface of the inner sheet 260a with sixth hot melt adhesives 66. It should be appreciated here that the waist elastic members 13 and/or the leg elastic members 14 may be provided in the form of strings or belts. It is also possible to form the front panel 270 and/or the rear panel 280 of liquid-pervious nonwoven fabrics which are elastically stretchable and contractible in the transverse direction X.

Referring to FIG. 2, a dimension of the inner member 1B in the longitudinal direction Y is shorter than that of the chassis 1A and a dimension of the inner member 1B in the transverse direction X is smaller than that of the inner sheet 260a in the center panel 260. Such inner member 1B includes an absorbent structure 20 in the form of fibrous batt layer and leakage-barrier cuffs 50 provided along both side edges of the absorbent structure 20 wherein the inner member 1B is shaped symmetrically about the longitudinal center line C-C.

The absorbent structure 20 comprises an aggregate 21 of liquid-absorbent materials for a core of the structure 20, a first wrapping sheet 31 adapted to cover an inner surface 21a of the aggregate 21 lying on a skin-facing side thereof (not shown), a second wrapping sheet 32 adapted to cover an outer surface 21b of the aggregate 21 lying on a garment-facing side thereof and a skin-contact sheet 33 lying between the first wrapping sheet 31 and the wearer's skin and bonded to the first wrapping sheet 31 by fourth hot melt adhesives 64. The aggregate 21 at least contains liquid-absorbent fibers as one of liquid-absorbent materials 21d and preferably contains the liquid-absorbent fibers of the amount ranging 20 to 100% by mass. The aggregate 21 may contain, in addition to the liquid-absorbent fibers, super-absorbent polymer particles of the amount ranging 0 to 80% by mass. Such super-absorbent polymer particles may be used in the form of a mixture with the liquid-absorbent fibers or in the form of a layer extending at any level of the aggregate 21 in the thickness direction R thereof. In some embodiments, 50 to 90% by mass of the super-absorbent polymer particles used preferably have a particle diameter ranging 300 to 600 micro meters. This is for the reason that the super-absorbent polymer particles having the particle diameter in the range of 300 to 600 micro meters are easy to handle in the course of making the absorbent structure 20 and suitable for the absorbent structure 20 required to absorb bodily fluids as quickly as possible. As the first wrapping sheet 31, a liquid-pervious sheet may be used. As such liquid-pervious sheet, for example, nonwoven fabrics, woven fabrics or perforated plastic films may be used and preferably nonwoven fabrics made of thermoplastic synthetic fibers such as spun bonded nonwoven fabrics (SB nonwoven fabrics), melt bonded nonwoven fabrics, melt blown nonwoven fabrics, spun bonded/melt blown/spun bonded nonwoven fabrics (SMS nonwoven fabrics) may be used wherein these types of nonwoven fabrics more preferably have a basis mass ranging 5 to 20 g/m² and are those chemically modified to become hydrophilic. As the second wrapping sheet 32, a liquid-pervious or liquid-impervious sheet such as nonwoven fabrics, woven fabrics or plastic films may be used. As the nonwoven fabrics, nonwoven fabrics made of thermoplastic synthetic fibers such as spun bonded nonwoven fabrics, melt bonded nonwoven fabrics, melt blown nonwoven fabrics or spun bonded/melt blown/spun bonded nonwoven fabrics may be used. These types of nonwoven fabrics preferably have a basis mass ranging 15 to 30 g/m². When the liquid-pervious second wrapping sheet 32 is used, this is preferably the sheet 32 chemically modified to become hydrophilic. The skin-contact sheet 33 is preferably formed of a nonwoven fabric made of thermoplastic synthetic fibers and chemically modified to become hydrophilic. The absorbent structure 20 comprising the aggregate 21 covered with the first and second wrapping sheets 31, 32 made of such nonwoven fabrics in the place of usually used tissue papers advantageously simplifies the production process and reduces the number of kinds of stock material to be used. The skin-contact sheet 33 serves to make a texture of the absorbent structure soft and at the same time to prevent the liquid-absorbent material from falling off the first wrapping sheet 31, This skin-contact sheet 33 may be formed of a nonwoven fabric made of thermoplastic synthetic fibers chemically modified to become hydrophilic. As an example, spun bonded nonwoven fabrics made of crimped conjugate fibers and having a basis mass ranging 5 to 20 g/m². As examples of conjugate fibers, eccentric core-in-sheath type conjugate fibers consisting of polyethylene as the sheath component and polyester as the core component, eccentric core-in-sheath type conjugate fiber consisting of polyethylene as the sheath component and polypropylene as the core component, and a mixture of these core-in-sheath type conjugate fibers can be enumerated.

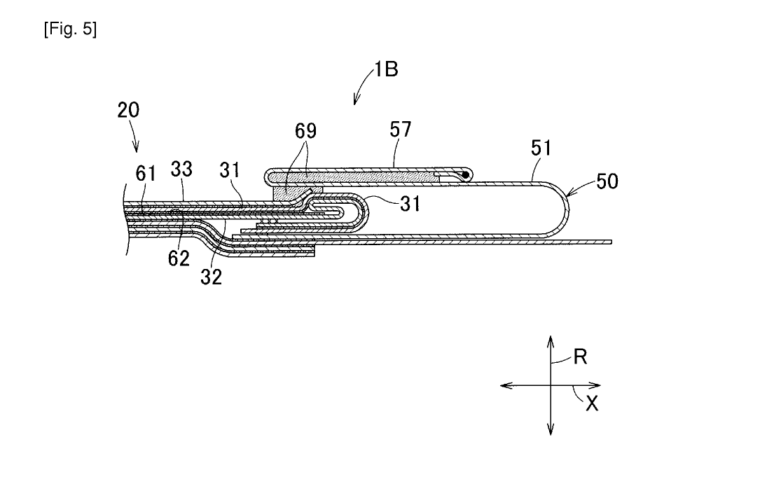

The leakage-barrier cuffs 50 are formed of strips 51 made of nonwoven fabrics and/or plastic films folded in Z-shape or inverted Z-shape and each of the cuffs 50 has a fixed edge 53 and a free edge 52 (See FIG. 4). The free edge 52 is folded to form a sheath within which an elastic string 56 is attached under tension. Opposite ends 57 of the leakage-barrier cuff 50 are bonded to at least one of the skin-contact sheet 33 and the first wrapping sheet 31 with ninth hot melt adhesives 69 (See FIG. 5). The strip 51 is preferably liquid-impervious or breathable and liquid-impervious.

Referring to FIG. 4, the aggregate 21 in the absorbent structure 20 has laterals (lateral surfaces) 21c opposed to each other in the transverse direction X and extending in the longitudinal direction Y. The inner surface 21a and the outer surface 21b become contiguous to each other along these laterals 21c. The first wrapping sheet 31 is bonded to the inner surface 21a of the aggregate 21 with first hot melt adhesives 61 to define a first extended portion 31a extending outward from the lateral 21c of the aggregate 21. The second wrapping sheet 32 is bonded to the outer surface 21b of the aggregate 21 with second hot melt adhesives 62 and extends upward in the thickness direction R to define a second extended portion 32a. The second extended portion 32a covers the lateral 21c also and further extends outward from the lateral 21c of the aggregate 21. These first and second extended portions 31a, 32a are put flat and bonded together with hot melt adhesives 61 applied to the first wrapping sheet 31 to define a laminated region 35 in which the first and second wrapping sheets 31, 32 are laminated together. This laminated region 35 extends downward from above in the thickness direction R. The second wrapping sheet 32 in the laminated region 35 is folded at a top portion 32b as viewed in FIG. 4 and extends in the inverted direction from this top portion. In a peripheral part 37 of the laminated region 35 extending downward, the segment of the second wrapping sheet 32 included therein is bonded to the second wrapping sheet 32 covering the aggregate 21 with third hot melt adhesives 63. Such laminated region 35 covers a part of the outer surface 21b and the lateral 21c of the aggregate 21 from outside the second wrapping sheet 32. The laminated region 35 is not bonded to a section of the second wrapping sheet 32 lying between the top portion 32b and third hot melt adhesive 63. With this arrangement, the laminated region 35 is spaced from a region of the second wrapping sheet 32 in which the second wrapping sheet 32 covers the lateral 21c in the transverse direction X as to form a void space 36 therebetween.

Referring also to FIG. 4, the skin-contact sheet 33 is bonded to the first wrapping sheet 31 with fourth hot melt adhesives 64. The strip 51 forming the leakage-barrier cuff 50 is folded in the inverted Z-shape and the portion of the leakage-barrier cuff 50 defining the fixed edge 53 is bonded to the first wrapping sheet 31 in the laminated region 35 with fifth hot melt adhesives 65. An imaginary line exemplarily shows a position of the leakage-barrier cuff 50 when the diaper 1 is put on the wearer's body and the inner member 1B is curved in the front-back direction Y (See FIG. 1). With the diaper 1 put on the wearer's body, the inner member 1B is curved under contraction of the elastic member 56 between the opposite ends 57 of the leakage-barrier cuff 50. Thereupon the leakage-barrier cuff 50 having been collapsed is expanded as indicated by the imaginary line and the free edge 52 of the leakage-barrier cuff 50 gets close to or comes in contact with the wearer's skin. In the case of the inner member 1B not required to prevent sideways-leakage of bodily fluids out of the diaper 1 by use of the leak-barrier cuff 50, it is possible to implement the present invention in the form of the diaper 1 without the leakage-barrier cuffs 50. It is also possible to implement the absorbent structure 20 without the skin-contact sheet 33 provided in the illustrated embodiment so far as the first wrapping sheet 31 can prevent the liquid-absorbent fiber and/or the super-absorbent polymer particles from falling off. Furthermore, in the absorbent structure 20, if a dimension of the laminated region 35 in the transverse direction X overlapping the second wrapping sheet 32 is sufficiently large, use of third hot melt adhesives 63 may be eliminated. Preferably, third hot melt adhesives 63 bond the laminated region 35 and the second wrapping sheet 32 continuously along the peripheries of the absorbent structure.

Referring again to FIG. 4, in a lower region of the inner member 1B, the inner sheet 260a, the intermediate sheet 260b and the outer sheet 260c constituting the center panel 260 are shown. The inner sheet 260a is bonded to the second wrapping sheet 32 and the leakage-barrier cuff 50 near its fixed edge 53. A side edge 81 of the inner sheet 260a extends outward from the leakage-barrier cuff 50 folded in the inverted Z in the transverse direction X and defines a side edge of the crotch region 6 in the diaper 1 and functions as a flap surrounding the wearer's leg. The intermediate sheet 260b is bonded to the inner sheet 260a with seventh hot melt adhesives 67. The intermediate sheet 260b functions also to prevent bodily fluids once absorbed by the absorbent structure 20 from leaking out of the diaper 1 through the second wrapping sheet 32. The intermediate sheet 260b is preferably formed of a plastic film and its dimension in the transverse direction X is preferably sufficient to overlap the laminated region 35 at least via the inner sheet 260a. The outer sheet 260c is bonded to the intermediate sheet 260b or to the intermediate sheet 260b and the inner sheet 260a. The outer sheet 260c functions to prevent the intermediate sheet 260b from coming in direct contact with the wearer's skin and is preferably formed of a nonwoven fabric having a comfortable texture. A dimension of the outer sheet 260c in the transverse direction X is preferably the same as or larger than that of the intermediate sheet 206b. When the second wrapping sheet 32 in the absorbent structure 20 is formed of a liquid-impervious sheet such as a liquid-impervious plastic film or a breathable but liquid-impervious plastic film, the center structure 260 may be implemented without the intermediate sheet 260b or without the intermediate sheet 260b and the outer sheet 260c.

Referring to FIG. 5, the line V-V lies outside a rear end (not shown) of the aggregate 21 as viewed in the longitudinal direction Y of the developed diaper 1a. At the opposite ends 57 of the leakage-barrier cuff 50 in the inner member 1B, the strip 51 folded in the inverted Z-shape is bonded together with ninth hot melt adhesives 69 and each of the ends 57 adhesively collapsed in this manner is bonded to at least one of the skin-contact sheet 33 and the first wrapping sheet 31. These ends 57 are fixed to the absorbent structure 20 and can not be expanded even when the diaper 1 is put on the wearer's body. The first wrapping sheet 31 and the second wrapping sheet 32 overlap each other and bonded to each other with first hot melt adhesives 61 and second hot melt adhesives 62 applied to these wrapping sheets 31, 32, respectively.

In the absorbent structure 20 of the diaper 1 constructed as has been described hereinabove, the aggregate 21 comprises the fibers having a relatively short fiber length such as fluff pulp fibers and the particles having a relatively small particle diameter such as super-absorbent polymer particles of which the particle diameter is 600 micro meters or less. With such construction, even if it is impossible for the second wrapping sheet 32 covering the laterals 21c of the aggregate 21 to prevent those fibers and particles from falling off, the problem can be overcome by the presence of the laminated regions 35 outboard of the second wrapping sheet 32. The respective laminated regions 35 are adapted to define the void spaces 36 between the respective laminated regions 35 and the second wrapping sheet 32 and thereby to prevent the fibers and/or the particles having fallen through the second wrapping sheet 32 from coming in contact with the wearer's skin and/or disfiguring the diaper 1. Certainly there is possibility, for example, under the effect of the wearer's body weight that the fibers and/or the particles constituting the aggregate 21 might fall through fine pores such as fiber interstices of the second wrapping sheet 32 or breaks of plastic film when the second wrapping sheet 32 is formed of a plastic film. However, the fibers and/or the particles having fallen through the second wrapping sheet 32 are received by the respective void spaces 36. In consequence, the possibility that the fibers and/or the particles might fall through the void spaces is effectively restricted. The presence of the laminated regions 35 also contributes to the effect of the respective void spaces 36. Specifically, each of the laminated regions 35 comprises the first and second wrapping sheets 31, 32 overlapping each other and bonded together, preventing those fibers and/or particles from falling out of the absorbent structure 20. In the case of the absorbent structure 20 according to the illustrated embodiment, the skin-contact sheet 33 overlaps the first wrapping sheet 31 and such arrangement effectively prevents the fibers and/or the particles constituting the aggregate 21 from fallen through the first wrapping sheet 31. The center panel 260 bonded to the absorbent structure 20 further improves the effect of preventing the fibers and/or the particles constituting the aggregate 21 from falling out of the diaper 1 through the second wrapping sheet 32.

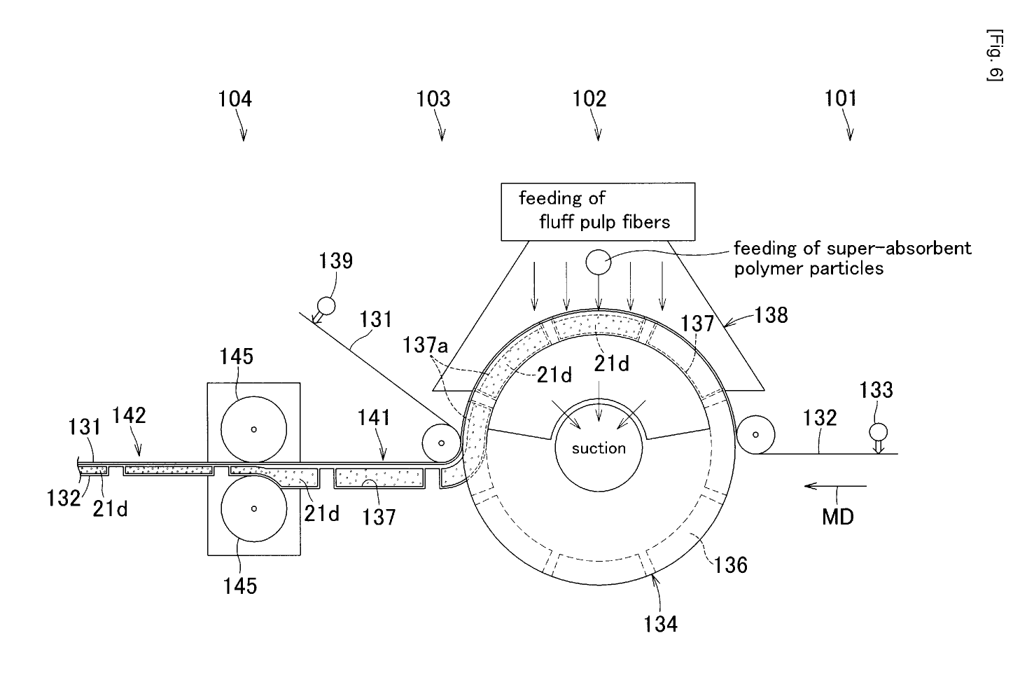

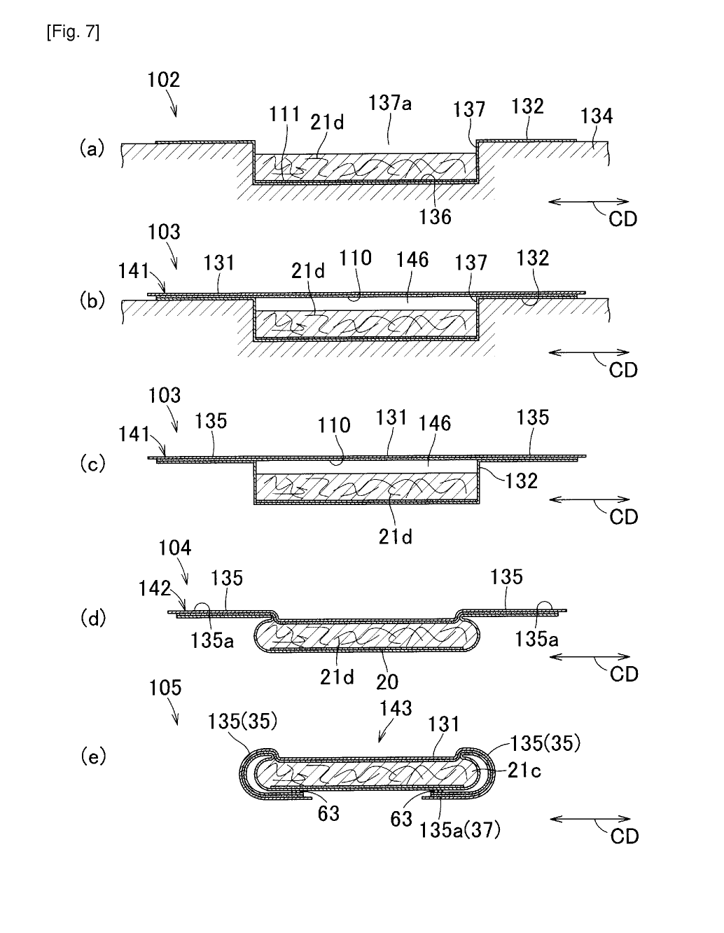

Steps in a process for making the absorbent structure 20 of FIG. 2 are exemplarily illustrated in FIGS. 6 and 7. In a first step 101 in FIG. 6, second web 132 corresponding to the second wrapping sheets 32 being contiguous one to another is fed from the upstream in a machine direction MD and coated by second nozzle 133 with first hot melt adhesives 111 at given regions (See FIG. 7) thereof. In a second step 102, the second web 132 is transferred onto a peripheral surface of a rotary drum 134. The second web 132 turns in the machine direction MD together with the rotary drum 134, being sucked into depressions 136 formed in the peripheral surface under a suction effect directed from the peripheral surface to a center of the rotary drum 134 in the course of turning and it is thereby formed with liquid-absorbent material receptacles 137 each shaped in accordance with the depressions 136. Also in the second step 102, the second web 132 having been formed with the liquid-absorbent material receptacles 137 runs into a liquid-absorbent material feeding section 138 wherein a predetermined quantity of the liquid-absorbent materials 21d, for example, fluff pulp fibers and super-absorbent polymer particles are fed into each of the receptacles 137. In the section 138, fluff pulp fibers and super-absorbent polymer particles may be fed to be mixed within the receptacle 137 or to form laminar structure within the receptacle 137. The second web 132 leaving the section 138 runs into a third step 103. In the third step 103, first web 131 corresponding to the first wrapping sheets 31 being contiguous one to another is coated by first nozzle 139 with tenth hot melt adhesive 110 at given regions (See FIG. 7) thereof, and is then put flat and bonded together with the second web 132 via the tenth hot melt adhesives 110. The first web 131 has a width dimension substantially the same as that of the second web 132 and is bonded to the second web 132 along front and rear ends as viewed in the machine direction MD and laterals extending in a cross direction CD (See FIG. 7) orthogonal to the machine direction MD to obtain a first composite web 141 wherein the first web 131 covers respective openings 137a of the receptacles 137. In a fourth step 104, the first composite web 141 is compressed in its thickness direction to obtain a second composite web 142 in which the thickness of the liquid-absorbent materials 21d in the respective receptacles is appropriately regulated.

It should be noted that, in FIG. 7, the web in the respective steps are shown in sectional views taken in the cross direction which is orthogonal to the machine direction MD in FIG. 6. FIG. 7(a) shows the second web 132 and the liquid-absorbent material 21d in FIG. 6. The drum 134 is formed with the depressions 136 and the second web 132 is formed with the receptacles 137 shaped in accordance with those of the depressions 136. Each of the receptacles 137 is coated on inner surface of its bottom with eleventh hot melt adhesives 111 and the liquid-absorbent materials 21d retained on the bottom of the receptacle 137 is bonded to the inner surface of the bottom via the eleventh hot melt adhesives 111. Within the receptacle 137, a quantity of the liquid-absorbent materials 21d free from apprehension of spilling out of the receptacle 137 is retained. FIG. 7(b) shows the first and second webs 131, 132 and the liquid-absorbent materials 21d in the third step 103 illustrated by FIG. 6. The first web 131 overlaps the second web 132 outside the receptacles 137 and is bonded to the second web 132 with tenth hot melt adhesives 110. The first web 131 closes the openings 137a of the respective receptacles 137 to leave gaps 146 between the first web 131 and the respective liquid-absorbent materials 21d. FIG. 7(c) shows the first composite web 141 leaving the peripheral surface of the drum 134 to the fourth step 104 illustrated by FIG. 6. The first composite web 141 is formed with horizontal laminated regions 135 extending in the cross direction CD on both sides of the receptacles 137. The horizontal laminated region 135 comprises the first and second webs 131, 132 bonded to each other with tenth hot melt adhesives 110 wherein the first web 131 extends in the cross direction CD from the side edges of the second web 132. FIG. 7(d) shows second composite web 142 obtained by compressing the first composite web 141 in the fourth step 104. In the second composite web 142, the gaps 146 formed in the first composite web 141 disappear and the liquid-absorbent material 21d is compressed to a desired thickness to form the aggregate 21 shown by FIG. 4. FIG. 7(e) shows a fifth step 105 subsequent to the fourth step 104 illustrated by FIG. 6. In the fifth step 105, the second web 132 constituting the second composite web 142 is coated in desired regions thereof with third hot melt adhesives 63 (See FIG. 4), then the second composite web 142 is let pass through guide means (not shown) serving to curve the horizontal laminated regions 135 along the laterals 21c of the aggregate 21. Then respective distal portions 135a of the horizontal laminated regions 135 are bonded to the second web 132 via third hot melt adhesives 63 to obtain third composite web 143. While not illustrated in FIG. 7 for simplification, the web to form the skin-contact sheet 33 is attached to the first web 131 in the third composite web 143 with fourth hot melt adhesives 64 (See FIG. 4). The third composite web 143 comprises the aggregates 21 intermittently arranged in the machine direction MD and the third web may be cut off between each pair of the adjacent aggregates 21, 21 to obtain the individual absorbent structures 20 as shown by FIG. 4. It should be appreciated here that the second web 132 used to obtain the absorbent structures 20 by the process illustrated by FIGS. 6, 7 is preferably formed of breathable nonwoven fabrics or breathable perforated plastic films. The breathable second web 132 may be adopted to accumulate the liquid-absorbent materials 21d in the receptacles 137 under the suction effect. In this way, the laminated regions 35 in the absorbent structure 20 as shown by FIG. 4 can be obtained from the horizontal laminated regions 135 in FIG. 7 and the peripheral portions 37 of the respective laminated regions 35 can be the distal portions 135a in FIG. 7. The tenth hot melt adhesives 110 corresponds to the first hot melt adhesives 61 in the absorbent structure 20 and the eleventh hot melt adhesives 111 corresponds to the second hot melt adhesive 62 in the absorbent structure 20.

The terms “first wrapping sheet” and “second wrapping sheet” in the description of the present invention and the embodiments thereof are used merely for the purpose of identifying them on the basis of configurations thereof and regions in which these sheets are used and not for the purpose of ranking them on the basis of priority. This is true for the hot melt adhesive. The terms “first, second, third, . . . hot melt adhesives” also used herein merely for the purpose of identifying them on the basis of regions in which hot melt adhesives is used but neither for the purpose of identifying them on the basis of priority ranking thereof nor for the purpose of identifying them on the basis of composition and/or property thereof. It should be noted that the hot melt adhesives is an example of means to bond the members and, depending on embodiments of the present invention, hot melt adhesives may be replaced by the other adhesive or pressure-sensitive adhesives or two or more members may be heat- or ultrasonically welded together.

The absorbent structure according to the illustrated embodiment and the other embodiments of the present invention may be used not only for the illustrated diaper 1 but for diapers having numerous different constructions, and also for other bodily fluid-absorbent wearing articles such as pant-type diapers, open-type diapers, toilet-training pants, incontinent pants, menstruation napkins and urine-absorbent pads.

The first aspects of the present invention described above may be arranged in at least the following items:

(i) An absorbent structure comprising an aggregate of liquid-absorbent materials covered with a liquid-pervious first wrapping sheet lying on a skin-facing side and a liquid-pervious or liquid-impervious second wrapping sheet lying on a garment-facing side, wherein: the absorbent structure has a longitudinal direction, a transverse direction and a thickness direction being orthogonal one to another and includes at least liquid-absorbent fiber as the liquid-absorbent material; the aggregate includes an inner surface lying on the side of the first wrapping sheet as viewed in the thickness direction, an outer surface lying on a side of the second wrapping sheet and lateral surfaces extending in the longitudinal direction on both sides as viewed in the transverse direction to connect the inner surface with the outer surface; the first wrapping sheet covers the inner surface and includes first extended portions extending in the transverse direction beyond the lateral surfaces to lie outboard of the aggregate and the second wrapping sheet covers the outer surface and the lateral surfaces and includes second extended portions extending in the transverse direction beyond the lateral surfaces so as to lie outboard of the aggregate; the first and second wrapping sheets are put flat and bonded together in the first and second extended portions to form laminated regions; and the laminated regions are curved downward as viewed in the thickness direction to cover the lateral surfaces and part of the outer surface, from the outside of the second wrapping sheet.

(xiv) A bodily fluid-absorbent wearing article comprising the absorbent structure as recited above.

One or more aspect of the present invention described in the above items (i) and (xiv) may provide one or more of the following advantageous effects:

(a) even if the liquid-absorbent material used by the absorbent structure leaks out through the regions of the second wrapping sheet covering the laterals of the absorbent structure, the presence of the laminated regions serves to prevent such amount of the liquid-absorbent material having leaked out through the second wrapping sheet from further leaking out of the absorbent structure and coming in contact with the wearer's skin.

Additionally, one or more of the following embodiments are provided in accordance with further aspects:

(ii) The laminated regions are not bonded to regions of the second wrapping sheet covering the laterals but bonded to regions of the second wrapping sheet covering the outer surface. Put differently, the laminated regions are bonded only to the regions of the second wrapping sheet covering the outer surface.

(iii) The laminated regions are bonded to the garment-facing side of said absorbent structure along their peripheries.

(iv) The laminated regions are each bonded to the garment-facing side of said absorbent structure along a continuous longitudinally extending bond line.

(v) A portion of each of the laminated regions covering a lateral surface of the aggregate is spaced transversely outwardly from the lateral surface so as to form a void between the portion of the second wrapping sheet covering the lateral surface and the laminated region.

(vi) The void is closed by the bond that bonds the laminated region to the garment-facing side of said absorbent structure.

(vii) The void extends the length of the aggregate in the longitudinal direction.

(viii) The second wrapping sheet is liquid-impervious.

(ix) The second wrapping sheet is liquid-pervious.

(x) The absorbent structure is free of tissue paper, the first and second wrapping sheets are in direct contact with the liquid-absorbent materials.

(xi) The first and second wrapping sheets are formed of liquid-pervious nonwoven fabrics.

(xii) A liquid-pervious skin-contact sheet lying between regions of the first wrapping sheet covering the inner surface and the wearer's skin is bonded to the regions of the first wrapping sheet covering the inner surface.

(xiii) The liquid-absorbent materials include super-absorbent polymer particles and 50 to 90% by mass of the super-absorbent polymer particles have a particle diameter ranging 300 to 600 micro meters.

(xv) The bodily-fluid absorbent wearing article is selected from the group including pant-type diapers, open-type diapers, toilet-training pants, incontinent pants, menstruation napkins and urine-absorbent pads.

According to the embodiments in the above (ii) to (xiii) and (xv), the advantageous effect(s) set forth at (a) is/are better ensured. It should be noted that features of these embodiments may be taken in isolation or in combination with one another. Further advantageous effects of the respective embodiments may be obtained as discussed in the respective related descriptions.

1. An absorbent structure comprising an aggregate of liquid-absorbent materials covered with a liquid-pervious first wrapping sheet lying on a skin-facing side and a liquid-pervious or liquid-impervious second wrapping sheet lying on a garment-facing side, wherein:
said absorbent structure has a longitudinal direction, a transverse direction and a thickness direction being orthogonal one to another and includes at least liquid-absorbent fiber as said liquid-absorbent material;
said aggregate includes an inner surface lying on the side of said first wrapping sheet as viewed in said thickness direction, an outer surface lying on a side of said second wrapping sheet and lateral surfaces extending in said longitudinal direction on both sides as viewed in said transverse direction to connect said inner surface with said outer surface;
said first wrapping sheet covers said inner surface and includes first extended portions extending in said transverse direction beyond said lateral surfaces so as to lie outboard of said aggregate and said second wrapping sheet covers said outer surface and said lateral surfaces and includes second extended portions extending in said transverse direction beyond said lateral surfaces so as to lie outboard of said aggregate;
said first and second wrapping sheets are put flat and bonded together in said first and second extended portions to form laminated regions; and
said laminated regions are curved downward as viewed in said thickness direction to cover said lateral surfaces and part of said outer surface of said aggregate, from the outside of said second wrapping sheet.

2. The absorbent structure defined by claim 1, wherein said laminated regions are bonded only to regions of said second wrapping sheet covering said outer surface.

3. The absorbent structure defined by claim 1, wherein said laminated regions are bonded to the garment-facing side of said absorbent structure along their peripheries.

4. The absorbent structure defined by claim 2, wherein the laminated regions are each bonded to the garment-facing side of said absorbent structure along a continuous longitudinally extending bond line.

5. The absorbent structure defined by claim 2, wherein a portion of each of the laminated regions covering a lateral surface of the aggregate is spaced transversely outwardly from the lateral surface so as to form a void between the portion of the second wrapping sheet covering the lateral surface and the laminated region.

6. The absorbent structure defined by claim 5, wherein the void is closed by the bond that bonds the laminated region to the garment-facing side of said absorbent structure.

7. The absorbent structure defined by claim 5, wherein the void extends the length of the aggregate in the longitudinal direction.

8. The absorbent structure defined by claim 1, wherein said second wrapping sheet is liquid-impervious.

9. The absorbent structure defined by claim 1, wherein said second wrapping sheet is liquid-pervious.

10. The absorbent structure defined by claim 1, wherein said absorbent structure is free of tissue paper, said first and second wrapping sheets are in direct contact with the liquid-absorbent materials.

11. The absorbent structure defined by claim 1, wherein said first and second wrapping sheets are formed of liquid-pervious nonwoven fabrics.

12. The absorbent structure defined by claim 1, wherein a liquid-pervious skin-contact sheet lying between a region of said first wrapping sheet covering said inner surface and said wearer's skin is bonded to said region of said first wrapping sheet covering said inner surface.

13. The absorbent structure defined by claim 1, wherein said liquid-absorbent materials includes super-absorbent polymer particles and 50 to 90% by mass of said super-absorbent polymer particles have a particle diameter ranging 300 to 600 micro meters.

14. A bodily fluid-absorbent wearing article comprising said absorbent structure defined by claim 1.

15. The bodily fluid-absorbent wearing article defined by claim 14, wherein the bodily-fluid absorbent wearing article is selected from the group including pant-type diapers, open-type diapers, toilet-training pants, incontinent pants, menstruation napkins and urine-absorbent pads.
